(19) United States
(12) Patent Application Publication
(10) Pub. No.: US 2021/0337995 A1
(43) Pub. Date: Nov. 4, 2021

(54) Furniture Having an Integrated Disinfecting Chamber

(71) Applicant: Daniel Maurer, Chicago, IL (US)

(72) Inventor: Daniel Maurer, Chicago, IL (US)

(21) Appl. No.: 16/865,981

(22) Filed: May 4, 2020

(51) Int. Cl. A47G 29/30; A61L 2/20; A61L 2/10

(52) U.S. Cl. CPC A47G 29/30; A61L 2/26; A61L 2/10; A61L 2/202

(57) ABSTRACT

An item of furniture, such as a bench, cabinet, desk, chest or sideboard, having an appearance suitable to be located near the door of a home, contains integrated within it a disinfection chamber suitable for disinfecting home-delivery packages. The disinfection chamber may use disinfecting light or ozonated air or both.

### CROSS-REFERENCE TO RELATED APPLICATIONS

NA

### STATEMENT REGARDING FEDERALLY SPONSORED RESEARCH OR DEVELOPMENT

NA

### THE NAMES OF THE PARTIES To A MINT RESEARCH AGREEMENT

NA

### REFERENCE TO COMPACT DISC

NA

### STATEMENT REGARDING PRIOR DISCLOSURES

NA

### BACKGROUND OF THE INVENTION

My invention relates to the field of Furniture.

It is common to receive packages delivered at home, typically at an exterior door near the street. Home delivered packages have often been handled by many people in the shipping process. To prevent the spread of disease, the recipients may want a simple and convenient method to reduce or eliminate pathogens left on the package from handling.

Disinfection equipment is commonly known and used in the medical and personal care fields to sterilize equipment and materials, such as with heat, steam, ultraviolet radiation or chemical disinfectants. Disinfection equipment is less common in the home setting. Small home disinfection equipment is available for sterilization of toothbrushes, dental appliances, telephones and CPAP equipment. However, these devices are not convenient for use to disinfect packages delivered to the home. They are generally too small for disinfecting home-delivery packages. Further, they look clinical and inappropriate for use near the formal entry to a home.

### BRIEF SUMMARY OF THE INVENTION

What is needed is a disinfecting system that is sized appropriately to disinfect ordinary home delivery packages and that is aesthetically designed to be appropriate inside a home near an exterior door such as the front door, so that packages can be disinfected near the point of delivery with minimal handling before disinfection.

My invention is an article of manufacture comprising:
a) An item of furniture having an appearance suitable to be located near the door of a home, and
b) integrated within the item of furniture, a disinfection chamber suitable for disinfecting home-delivery packages.

The furniture of the present invention can be located inside a home near an exterior door of a home and fit into the desired appearance of the area, and it allows the convenient disinfection of packages with minimal handling and without taking the packages through the rest of the home and without placing the packages in contact with other surfaces in the home.

### BRIEF DESCRIPTION OF THE SEVERAL VIEWS OF THE DRAWING

NA

### DETAILED DESCRIPTION OF THE INVENTION

The invention comprises an item of furniture that is suitable to appear near the doorway of a home. The item of furniture must have internal space large enough to hold the integrated disinfection chamber. Examples of suitable furniture are a bench, cabinet, chest, desk or sideboard. The most preferred example is a bench or chest.

Suitable furniture can be made from any strong, durable material such as wood, engineered wood (such as plywood, particle board or chip board), metal or plastic. The structural elements preferably comprise wood or engineered wood. The external front-facing surfaces of the furniture preferably comprise finished wood, wood laminate, wood veneer, wicker, marble, quartz and/or ceramic surfaces and more preferably comprise wood or wood laminate or veneers. The front-facing exterior of the furniture more preferably further contains decorative elements such as carving, fretwork, inlays and decorative hardware. The external appearance is preferably not the appearance of an appliance.

The invention must contain a disinfection chamber suitable for disinfecting home delivery packages integrally located within the item of furniture. The disinfection chamber preferably has an internal height of at least 30 cm, more preferably at least 45 cm and most preferably at least 60 cm. The disinfection chamber preferably has an internal depth of at least 30 cm, more preferably at least 45 cm and most preferably at least 60 cm. The disinfection chamber preferably has an internal width of at least 30 cm, more preferably at least 60 cm and most preferably at least 90 cm.

The disinfection chamber must contain sufficient walls and at least one door to fully enclose the items for disinfection and protect persons nearby during the disinfection process. Preferably, the door either locks when the disinfecting process is in progress or shuts off the disinfecting process when the door is not fully closed or both.

The disinfection chamber preferably disinfects items using a gaseous disinfecting chemical or light or both. The method of disinfection is preferably suitable for use by untrained people in the home. The most preferred gaseous disinfecting chemical is ozone. The most preferred disinfecting light is UVC light. Most preferably, the disinfection chamber disinfects using UVC light.

### Light Disinfection Chambers

Disinfection chambers that use light comprise:
a) one or more light sources that emit a disinfecting light and are positioned to illuminate the items to be disinfected;
b) one or more power sources wired to provide power to the light sources; and
c) one or more control mechanisms wired to control the emission of light from the light source by completing or interrupting the circuit between the light sources and the power source.

Light generating systems for disinfection chambers are described in the following references, which are incorporated herein by reference: Collard et al., Apparatus for Sanitizing Oral Appliances, U.S. Pat. No. 8,685,318 B2 (Apr. 1, 2014); Deshays et al, Methods, Systems, and Devices for High-Level Disinfection, U.S. Pat. No. 10,111,976 B2 (Oct. 30, 2018); Eckhardt, Method and Apparatus for Sterilizing Small Objects, U.S. Pat. No. 6,461,568 B1 (Oct. 8, 2002); Gaska et al., Ultraviolet-Based Sterilization, U.S. Pat. No. 9,061,082 B2 (Jun. 23, 2015); Hughes, UVC Sterilization Box Electronics Devices, US Patent Pub US 2106/0158395 A1 (Jun. 9, 2016); Shur et al., Ultraviolet System for Disinfection, U.S. Pat. No. 9,179,703 B2 (Nov. 10, 2015); and Yerby, Apparatus and Method for Sanitizing Articles, US Patent Pub US 2016/0206766 (Jul. 21, 2016).

The light source is typically a bulb or LED. Bulbs and LEDs to produce disinfecting light are commercially available. The source preferably generates ultraviolet light with a wavelength of about 185 nm to about 400 nm. The light more preferably has a wavelength of at least about 200 nm and most preferably has a wavelength of at least about 205 nm. The light more preferably has a wavelength of at most about 285 nm, and most preferably has a wavelength of at most about 270 nm. Ultraviolet light with a wavelength between about 205 nm to about 225 nm (“Far UV”) may be safer for human exposure, whereas ultraviolet light with a wavelength between about 250 nm and 270 nm may be more effective for quick disinfection.

The power source is preferably a simple plug to a wall socket, with any transformer necessary to adjust voltage for the light source.

Examples of suitable control mechanisms include a mechanical switch, a mechanical timer switch or digital controller. The control mechanism is more preferably a mechanical timer or a digital controller, and most preferably a digital controller. The control mechanism preferably further operates a lock to lock the door to the disinfection chamber during the disinfection process and/or shuts off the light sources when the door is open. The control mechanism preferably operates the light source for a time sufficient to effectively disinfect the items to be disinfected, and then shuts the light sources off.

The disinfection chamber that uses light preferably further comprises an internal lining that reflects the disinfecting light onto the items to be disinfected. Suitable reflecting materials for UV light are well known and include, for example, mirrors or reflective sheeting containing polytetrafluoroethylene (PTFE) or aluminum, aluminum foil or reflective paints. The internal lining more preferably comprises a mirror and most preferably comprises aluminum. The disinfection chamber may also contain other reflectors to more evenly distribute disinfecting light to the surface of the items to be disinfected.

The disinfection chamber that uses light preferably further comprises a support to hold the items to be disinfected off the floor of the chamber, so that disinfecting light can reach the bottom of the items to be disinfected. The support preferably comprises a mesh or perforated material or a material that permits substantial transmission of the disinfecting light. The support more preferably comprises a metal mesh or a UVC-transparent glass and most preferably comprises a metal mesh.

The arrangement of light sources, internal lining, reflectors and support in the disinfection chamber preferably illuminates substantially the entire surface of the items to be disinfected with a quantity of disinfecting light that is effective to substantially kill pathogens on the surface. The disinfection chamber preferably comprises light sources in at least 2 different locations and more preferable comprises light sources in at least 4 different locations. Optionally, the support or the light sources move to provide more even exposure to disinfecting light for the items to be disinfected.

Preferably, the disinfection chamber further comprises a ventilation system, such as an air pump and one or more vents, to prevent excess temperature in the disinfection chamber that may damage the items to be disinfected. The temperature in the disinfection chamber preferably does not exceed 20° C. over ambient temperature during the disinfection process, and more preferably does not exceed 10° C. over ambient temperature.

### Ozone Disinfection Chambers

Disinfection chambers that use ozone comprise:
a) An air inlet;
b) An air pump connected to the air inlet;
c) An ozone generator connected to the air pump and to the disinfection chamber, such that air moves from the air inlet through the ozone generator and then into the disinfection chamber;
d) One or more power sources to power the air pump and the ozone generator; and
e) One or more control mechanisms wired to control the air pump and the ozone generator by completing or interrupting the circuits from the power source(s) to the ozone generator and air pump.
   Disinfection chambers containing these elements are described in the following references, which are incorporated herein by reference: Bohman et al., Ozone Sanitizing System and Method, US Patent Publication US 2020/0000950 (Jan. 2, 2020); Kain et al., Ozone Sanitizing System, U.S. Pat. No. 9,327,040 B2 (May 3, 2016); Leyva et al., Devices, Systems and Methods for Treating Medical Devices having Passage Ways with Ozone Gas, U.S. Pat. No. 10,232,072 B2 (Mar. 19, 2019); Malkin et al., Ozone Applications for Disinfection, Purification and Deodorization, U.S. Pat. No. 6,391,259 B1 (May 21, 2002); Schwarz et al., Disinfecting Articles with Ozone, US Patent Publication US 2018/0207307 A1 (Jul. 26, 2018); Sham, Sanitizing Mailbox Apparatus and Method, US Patent Publication US 2003/0152480 A1 (Aug. 14, 2003); and Thomas et al., Method and Apparatus for Using Ozone as Disinfectant, U.S. Pat. No. 6,872,366 B2 (Mar. 29, 2005).

Suitable air pumps and ozone generators are commercially available. Preferably, the air pump is located between the air inlet and the ozone generator, so that the air pump is not exposed to air with raised levels of ozone.

The power source is preferably a simple plug to a wall socket, with any transformers necessary to adjust voltages for the air pump and the ozone generator.

Examples of suitable control mechanisms include a mechanical switch, a mechanical timer switch or digital controller. The control mechanism is most preferably a digital controller. The control mechanism preferably further operates a lock to lock the door to the disinfection chamber during the disinfection process and/or shuts off the air pump and the ozone generator when the door is open. The control mechanism preferably operates both the air pump and the ozone generator for a time sufficient to effectively disinfect the items to be disinfected, and then shuts off the ozone generator and operates the air pump for an additional time to substantially purge ozone from the disinfection chamber.

The disinfection chamber preferably further comprises an internal lining, gaskets and conduits made of ozone-resistant material, to reduce leakage of ozone from the disinfection chamber. Examples of ozone-resistant material include Hasteloy and Inconel steel, Teflon coated materials, silicone, chlorinated PVC and polycarbonate.

The disinfection chamber preferably further comprises an air outlet with an ozone removing means, to permit circulation of air through the chamber while reducing the release of ozone outside the disinfection chamber. Preferred ozone removing means include activated carbon beds and solid catalyst beds. The ozone removing means most preferably comprises activated carbon.

The disinfection chamber preferably further comprises a support to hold items off the floor of the disinfection chamber so that ozone gas can reach all sides of the package. The support is preferably a perforated, wire or mesh shelf comprising ozone-resistant material,

The control mechanism should operate the air pump and ozone generator for long enough raise and maintain the ozone content in the disinfection chamber to a level and for a time sufficient to substantially reduce pathogens on the item to be disinfected. The optimum level and time will vary based on a number of factors, including the efficiency of the ozone generating system and the size of the chamber. Preferably the ozone content in the disinfection chamber reaches at least 3 ppm, more preferably at least 5 ppm and most preferably at least 10 ppm.

For both light and ozone disinfection chambers:
a) Preferably, the disinfection chamber does not use liquid or flammable disinfectants.
b) Preferably, the disinfection chamber does not subject the packages to excess heat.

Preferably, the temperature in the disinfection chamber reaches no more than about 20° C. above the ambient temperature outside the furniture, and more preferably no more than 10° C. higher.

A preferred embodiment of the invention is illustrated by the following examples.

### EXAMPLE 1

### One Light Source

A commercially-available wooden seating bench with a fully enclosed internal storage chamber is obtained. The storage chamber has 4 wooden sides, a wooden floor and a hinged wooden lid that forms seat for the bench. The storage chamber has internal dimensions of about 18 inches deep by about 32 inches wide by about 20 inches high.

A hole for wiring is drilled in the back wall of the storage chamber about 6 inches below and to the left of the upper right-hand corner. A commercially-available light socket is installed in the chamber above the hole and wired to an ordinary wall-plug using a cord having a switch. The inside of the chamber (walls, floor and lid) is lined with commercially-available aluminum foil.

A free-standing wire shelf is placed in the bottom of the chamber.

A 30 watt “cob-style” UV-C LED light bulb (Model ACL30W-01-U, manufactured by Dongguan Zhao Peng Lighting Technology Co., Ltd.) is inserted in the light socket.

An object to be disinfected is placed on the shelf in the chamber. The object has UV indicator labels on four sides and the top (CON-TROL-CURE™ UV-C Intensity Labels N010-004). The lid of the closed and the light is turned on. The labels are checked after 5, 8 and 12 cumulative minutes of exposure. The labels start light yellow and turn dark blue-green to indicate exposure.
After 5 minutes, the label facing the light source is substantially blue-green, and the remaining labels are beginning the transition to blue-green.
After 8 minutes, the label facing the light source shows that it has received a UV exposure of at least 600 mJ/cm², the label away from the light source shows that it has received a UV exposure of at least 75 mJ/cm², and the remaining labels show that they have received a UV exposure of at least 150 mJ/cm².
After 12 minutes, the label facing the light source shows that it has received a UV exposure of at least 800 mJ/cm², the label away from the light source shows that it has received a UV exposure of at least 150 mJ/cm², and the remaining labels show that they have received a UV exposure of at least 600 mJ/cm².

### EXAMPLE 2

### Two Light Sources

In the article in Example 1, a second light socket is installed in the back wall of the disinfection chamber about 6 inches below and to the right of the upper left corner, using the same installation and wiring as the first light socket. A similar UV LED bulb is inserted.

I claim:

1. An article of manufacture comprising
(a) An item of furniture having an appearance suitable to be located near the door of a home, and
(b) integrated within the item of furniture, a disinfection chamber suitable for disinfecting home-delivery packages.

2. The article of claim 1 wherein the item of furniture is a bench, cabinet, desk, chest or sideboard.

3. The article of claim 1 wherein the front-facing external surfaces of the item of furniture comprise finished wood, wood laminate, wood veneer, wicker, marble, quartz or ceramic.

4. The article of claim 1 wherein the disinfection chamber further comprises
(a) one or more light sources that emit ultraviolet light and are positioned to illuminate the items to be disinfected;
(b) one or more power sources wired to provide power to the light sources; and
(c) one or more control mechanisms wired to control the emission of light from the light source by completing or interrupting the circuit between the light sources and the power source.

5. The article of claim 4 wherein the disinfection chamber further comprising an internal lining of ultraviolet reflective material.

6. The article of claim 5 wherein the disinfection chamber further comprises at least one support to hold items that are disinfected above the floor of the disinfection chamber and allow light to reach the bottom of the items.

7. The article of claim 6 wherein the disinfection chamber further comprises a ventilation system to control temperature in the disinfection chamber.

8. The article of claim 5 wherein the disinfection chamber has an internal height, width and depth of at least 30 cm.

9. The article of claim 8 wherein at least one dimension among the height, width and depth of the disinfection chamber is at least 60 cm.

10. The article of claim 5 wherein the item of furniture is a bench, cabinet, desk, chest or sideboard and the external front-facing surfaces of the furniture comprise finished wood, wood laminate, wood veneer, wicker, marble, quartz and/or ceramic surfaces.

11. The article of claim 1 wherein the disinfection chamber further comprises:
(a) An air inlet;
(b) An air pump connected to the air inlet;
(c) An ozone generator connected to the air pump and to the disinfection chamber, such that air moves from the air inlet through the ozone generator and then into the disinfection chamber;
(d) One or more power sources to power the air pump and the ozone generator; and
(e) One or more control mechanisms wired to control the air pump and the ozone generator by completing or interrupting the circuits from the power source(s) to the ozone generator and air pump.

12. The article of claim 11 wherein the disinfection chamber further comprises an internal lining of ozone resistant material.

13. The article of claim 12 wherein the disinfection chamber further comprises an air outlet having ozone-removing material.

14. The article of claim 12 wherein the disinfection chamber has an internal height, width and depth of at least 30 cm.

15. The article of claim 13 wherein at least one dimension among the height, width and depth of the disinfection chamber is at least 60 cm.

16. The article of claim 12 wherein the item of furniture is a bench, cabinet, desk, chest or sideboard the item is a bench, cabinet desk, chest or sideboard and the external front-facing surfaces of the furniture comprise finished wood, wood laminate, wood veneer, wicker, marble, quartz and/or ceramic surfaces.
